(19) United States
(12) Patent Application Publication
(10) Pub. No.: US 2024/0075009 A1
(43) Pub. Date: Mar. 7, 2024

(54) TREATMENT OF SUICIDALITY WITH PSILOCIN OR PSILOCYBIN

(71) Applicant: NEW YORK UNIVERSITY, New York, NY (US)

(72) Inventors: Gabrielle AGIN-LIEBES, San Francisco, CA (US); STEPHEN ROSS, New York, NY (US)

(21) Appl. No.: 18/260,359

(22) PCT Filed: Jan. 7, 2022
(86) PCT No.: PCT/US2022/011576

§ 371 Date: Jul. 5, 2023

Related U.S. Application Data
(60) Provisional application No. 63/135,101, filed on Jan. 8, 2021.

(51) Int. Cl. A61K 31/4045; A61K 31/675; A61P 25/00

(52) U.S. Cl. CPC A61K 31/4045; A61K 31/675; A61P 25/00

(57) ABSTRACT

The invention relates to a method of preventing or reducing suicidal ideation and/or desire for hastened death in a patient suffering from a life-threatening disease, the method comprising administering a psychedelic compound which is psilocin or a prodrug of psilocin, or a pharmaceutically acceptable salt thereof, to said patient. Kits useful in the method of the invention are also described.

### FIELD OF THE INVENTION

The present invention relates to a method of preventing or reducing suicidal ideation and/or preventing or reducing desire for hastened death in a patient suffering from a life-threatening disease, such as cancer.

### BACKGROUND OF THE INVENTION

There are approximately 48,000 and 804,000 deaths by suicide each year in the US and worldwide, respectively, making it one of the leading causes of death. There are multiple risk factors for completed suicide including: Caucasian ethnicity, middle-age, male, history of mental illness (especially major depressive disorder), alcohol or substance use disorders, feelings of hopelessness, social isolation, loss (i.e. relational, social, work, financial), and medical illnesses (especially life-threatening diseases).

Among medical illnesses, the diagnosis of cancer can be a severe stressor and is a known risk factor for increased suicidal ideation (SI) and completed suicides. Studies of completed suicide in cancer patients have reported prevalence rates up to four times greater than in the general population. The association between cancer and increased suicide risk may be mediated by advanced illness (e.g. poor prognosis, disease progression), psychiatric distress (depression in particular), existential distress (hopelessness and helplessness), and uncontrolled pain.

A more subtle manifestation of suicidal ideation in patients with advanced, life-threatening diseases such as cancer is the construct of desire for hastened death (DHD), the wish for a more rapid death than would naturally occur. DHD has been reported in 9-22% of patients with advanced or terminal cancer and is more common in palliative care settings.

A known intervention for the treatment of anxiety and depression in individuals with cancer is psychedelic-assisted psychotherapy with psilocybin (Griffiths et al (2016), “Psilocybin produces substantial and sustained decreases in depression and anxiety in patients with life-threatening cancer: A randomized double-blind trial”, J Psychopharmacol, 30: 1181-1197; Ross S et al (2016), “Rapid and sustained symptom reduction following psilocybin treatment for anxiety and depression in patients with life-threatening cancer: A randomized controlled trial”, J Psychopharmacol, 30: 1165-1180; Agin-Liebes et al (2020), “Long-term follow-up of psilocybin-assisted psychotherapy for psychiatric and existential distress in patients with life-threatening cancer”, J Psychopharmacol, 34: 155-166).

Psilocybin (4-phosphoryloxy-N,N-dimethyltryptamine) is a tryptamine serotoninergic psychedelic. The IUPAC name of psilocybin is [3-(2-dimethylaminoethyl)-1H-indol-4-yl] dihydrogen phosphate. The structure of psilocybin is shown below.

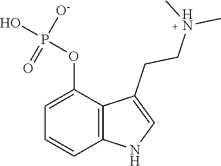

Psilocybin is metabolised in the body to psilocin (4-hydroxy-N,N-dimethyltryptamine), which exerts its effects primarily via SHT_{2A} agonism. The structure of psilocin is below.

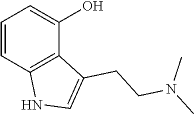

Treatments for anxiety and depression are not always effective at reducing suicidal ideation and/or hastened desire for death. In fact, it has been well known for many years that some anti-depressants, for instance selective serotonin reuptake inhibitors (SSRIs), can increase the risk of suicidality (e.g. Fergusson et al., (2005), “Association between suicide attempts and selective serotonin reuptake inhibitors: systematic review of randomised controlled trials”, British Medical Journal, 330). Since 2007, the US FDA have required a black box warning on antidepressants of all classes warning of an increased risk of suicidal thoughts and behaviours for certain age groups.

Hendricks P et al. (2015), “Psilocybin, psychological distress, and suicidality”, J Psychopharmacol, 29, 9 evaluates the associations of lifetime, recreational psilocybin use with past month psychological distress, past year suicidal thinking, past year suicidal planning, and past year suicide attempt in the United States adult population based on data from the National Survey on Drug Use and Health pooled across years 2008 to 2012. Cahart-Harris et al (2018), “Psilocybin with psychological support for treatment-resistant depression: six-month follow-up”, Psychopharmacology (Berl), 235: 399-408 reports the results from an open-label trial, in which individuals with treatment-resistant major depressive disorder received two doses, 10 mg and 25 mg, of psilocybin-assisted therapy. However, known treatments against mental disorders associated with depression and anxiety can have limited efficacy when administered to patients with life threatening diseases.

There remains the need for the development of an effective treatment for preventing or reducing suicidal ideation and/or hastened desire for death in patients suffering from life-threating diseases such as cancer.

### SUMMARY OF THE INVENTION

It is a finding of the present invention that administration of a psychedelic compound such as psilocybin is effective in preventing or reducing suicidal ideation and/or desire for hastened death in patients with life-threatening diseases such as cancer. Advantageously, it has been shown that psilocybin can cause a rapid reduction in suicidal ideation and desire for hastened death therefore providing an acute treatment for these indications. It has also been shown that long-term, sustained reduction in suicidal ideation and desire for hastened death is achieved with only a single dose of psilocybin, which means that it is not necessary for the patient to further complicate the pharmaceutical treatment regime for their life-threatening disease with chronic pharmacological interventions for reducing SI and or DHD.

The invention accordingly provides a method of preventing or reducing suicidal ideation and/or desire for hastened death in a patient suffering from a life-threatening disease, the method comprising administering a psychedelic compound which is psilocin or a prodrug of psilocin, or a pharmaceutically acceptable salt thereof, to said patient.

Also provided by the invention is a kit comprising: a psychedelic compound which is psilocin or a prodrug of psilocin, or a pharmaceutically acceptable salt thereof; and instructions for use of said psychedelic compound in a method of preventing or reducing suicidal ideation and/or desire for hastened death in a patient suffering from a life-threatening disease, the method comprising administering the psychedelic compound to said patient.

The invention further provides a psychedelic compound which is psilocin or a prodrug of psilocin, or a pharmaceutically acceptable salt thereof, for use in preventing or reducing suicidal ideation and/or desire for hastened death in a patient suffering from life-threatening disease.

The invention also provides a use of a psychedelic compound which is psilocin, or a prodrug of psilocin, or a pharmaceutically acceptable salt thereof, in the preparation of a medicament for use in preventing or reducing suicidal ideation and/or desire for hastened death in a patient suffering from a life-threatening disease.

### BRIEF DESCRIPTION OF THE DRAWINGS

FIGS. 1A-B show the mean (±SE) changes in primary outcome variables (DHD and SI) pre-crossover in two treatment groups (psilocybin-first n=6, niacin-first n=5) across the following time points: baseline, 8-hour post-dose, 2-weeks post-dose, and 7-weeks post-dose. The composite SI measure (BDI/BSI) reflects T-score values. Desire for Hastened Death (DHD) was not administered at the 8-hour post-dose and 7-weeks post-dose time points. Longitudinal within-subject effect sizes, represented as Cohen's d, are shown above and below the time points for the niacin-first group and psilocybin-first group, respectively. Asterisks indicate significant within-subject reductions relative to scores at baseline, *p<0.05, **p<0.01, ***p<0.001. †At 2-weeks post-dose 1 on the DHD there was a main effect of group, and pairwise comparisons indicated a between-group difference at this time point (p<0.05; Cohen's d=1.32). Note: There was no significant omnibus interaction of group x time within the mixed effect repeated measurement model for DHD.

FIGS. 2A-B show the mean (±SE) changes in primary outcome variables (DHD and SI) post-crossover at the following time points: Baseline (n=11), 6.5 months (parent study endpoint; n=8), 3.2 years (first follow-up; n=4), and 4.5 years (second follow-up; n=4). The composite SI measure (BDI/BSI) reflects T-score values and was not administered at the two long-term follow-up time points. Asterisks indicate significant within-subject differences relative to scores at baseline, *p<0.05, **p<0.01, ***p<0.001. Longitudinal within-subject effect sizes, represented as Cohen's d, are shown above time points.

### DETAILED DESCRIPTION OF THE INVENTION

As used herein, the term “about” means any value that the skilled person would appreciate is a reasonable variation of the value that is referred to by the term “about”. Typically, “about” means±10% or ±5%.

The method comprises administering a psychedelic compound which is psilocin or a prodrug of psilocin, or a pharmaceutically acceptable salt thereof. The psychedelic compound may accordingly be (i) psilocin or a pharmaceutically acceptable salt of psilocin or (ii) a prodrug of psilocin or a pharmaceutically acceptable salt of the prodrug of psilocin. In one embodiment, the psychedelic compound is a prodrug of psilocin or a pharmaceutically acceptable salt thereof.

A pharmaceutically acceptable salt of a compound is a salt formed with the compound and a non-toxic counter-ion which is suitable for administration to a patient. Pharmaceutically acceptable salts are well-known to the skilled person.

A prodrug of psilocin is a compound which is metabolised (or undergoes a biotransformation) to psilocin after it is administered to the patient. The prodrug of psilocin is typically an ester, carbonate ester, phosphate ester, amide or ether derivative of psilocin. Typically, the prodrug of psilocin is a phosphate ester of psilocin or a pharmaceutically acceptable salt thereof.

In one embodiment, the psychedelic compound is psilocybin or a pharmaceutically acceptable salt thereof. The invention accordingly provides a method of preventing or reducing suicidal ideation and/or preventing or reducing desire for hastened death in a patient suffering from a life-threatening disease, the method comprising administering a psychedelic compound which is psilocybin or a pharmaceutically acceptable salt thereof to said patient. In one embodiment, the psychedelic compound is psilocybin.

The psychedelic compound may be administered in any suitable form. Typically, the psychedelic compound is administered in a solid form. The psychedelic compound may be in amorphous or crystalline form. The psychedelic compound may be in the form of a solvate. The psychedelic compound may alternatively be administered as a solution.

The psychedelic compound may be administered as a compound which has been synthesised or which has been isolated from a natural source. Alternatively, the psychedelic compound may be administered as part of a natural source which comprises the psychedelic compound. In one embodiment, the method of the invention comprises administering a single dose of a natural source comprising the psychedelic compound. The natural source may be a mushroom of one of the following genera: Psilocybe, Gymnopilus, Panaeolus, Copelandia, Hypholoma, Pluteus, Inocybe, Conocybe, Panaeolina, Gerronema, Agrocybe, Galerina or Mycena. Typically, the natural source may be a mushroom of the Psilocybe genus, for example Psilocybe cubensis, Psilocybe cyanescens, Psilocybe semilanceata or Psilocybe azurescens.

The method is a method of preventing or reducing suicidal ideation and/or preventing or reducing desire for hastened death in a patient suffering from a life-threatening disease. The method may be a method of preventing or reducing suicidal ideation in a patient suffering from a life-threatening disease. The method may be a method of reducing suicidal ideation in a patient suffering from a life-threatening disease. The method may be a method of preventing or reducing desire for hastened death in a patient suffering from a life-threatening disease. The method may be a method of reducing desire for hastened death in a patient suffering from a life-threatening disease.

As used herein, preventing or reducing suicidal ideation includes preventing or reducing suicidal thinking, suicidal planning and/or suicide attempts. The patient may report a reduction in suicidal thinking and/or suicidal planning. The patient may make less frequent suicide attempts.

As used herein, preventing or reducing desire for hastened death includes preventing or reducing the desire for a more rapid death than would naturally occur. The patient may report a reduction in desire for a more rapid death than would naturally occur.

In one embodiment, the patient suffering from a life-threatening disease has been identified as being in need of treatment to prevent or reduce suicidal ideation and/or prevent or reduce desire for hastened death. Accordingly, the method of the invention may include a step of assessing the level of suicidal ideation and/or desire for hastened death in the patient prior to administering the psychedelic compound to said patient. In one embodiment, the patient has indicated that he or she is suffering from suicidal ideation and/or desire for hastened death.

Suicidal ideation may be measured using a composite test comprising elements from the Beck Depression Inventory-II (BDI-II, Beck et al (1988), “Psychometric properties of the Beck Depression Inventory: Twenty-five years of evaluation”, Clin Psych Rev, 8: 77-100) and the Brief Symptom Inventory (BSI; Derogatis 1993). In the BDI, Item #9 queries suicidal ideation with the following options: 0=I don't have any thoughts of killing myself; 1=I have thoughts of killing myself, but I would not carry them out; 2=I would like to kill myself; 3=I would kill myself if I had the chance. In the BSI, item #9 (“Thoughts of ending your life”) also correlates to suicidal ideation, and is measured on a Likert scale: 0=Not at all; 1=Little; 2=Moderately; 3=Quite a bit; 4=Extremely. The aggregate composite suicidal ideation score is calculated by adding the scores from BDI-II item #9 to BSI Item #9. The composite score may be calculated by computing Z-scores for each item and summing them, and then the composite Z-scores may be transformed into standardized T-scores with a range of 0 to 100 (Song et al., 2013). Higher scores indicate higher SI.

Accordingly, in the method of the invention a composite suicidal ideation score of the patient may be reduced after administration of the psychedelic compound. Typically, a composite suicidal ideation score of the patient is reduced by at least 20%, at least 30%, at least 40%, at least 50% or at least 75% after administration of the psychedelic compound. In one embodiment, a composite suicidal ideation score of the patient is reduced by at least 30% after administration of the psychedelic compound. In one embodiment, a composite suicidal ideation score of the patient after administration of the psychedelic compound is less than 50, less than 45 or less than 40.

The patient to be treated may have a composite suicidal ideation score of at least 60 or at least 70 prior to treatment. The composite suicidal ideation score of the patient may be measured prior to the treatment.

Desire for hastened death may be measured using the schedule of attitudes towards hastened death (SAND) (Rosenfeld 2000). The SAND is a 20-item true/false measure of desire for hastened death, which has been validated in patients with cancer. Alternatively, DHD can be measured using the loss of meaning factor from the Demoralization Scale (Kissane et al. (2004)). In particular, a composite desire for hastened death score can be created from the following five items from the loss of meaning factor, as measured on a Likert scale from zero to four: “Life is no longer worth living”, “I would rather not be alive”, “My life seems to be pointless”, “My role in life has been lost”, and “There is no purpose to the activities in my life”.

Accordingly, in the method of the invention a composite desire for hastened death score of the patient may be reduced after administration of the psychedelic compound. Typically, a composite desire for hastened death score of the patient is reduced by at least 20%, at least 40%, at least 60% or at least 80% after administration of the psychedelic compound. A composite desire for hastened death score of the patient may be reduced by at least 40% or at least 60% after administration of the psychedelic compound. In one embodiment, a composite desire for hastened death score of the patient is reduced by at least 40% after administration of the psychedelic compound. In one embodiment, a composite desire for hastened death score of the patient after administration of the psychedelic compound is less than 4.

The patient to be treated may have a composite desire for hastened death score of at least 4, at least 5 or at least 6 prior to treatment. The composite desire for hastened death score of the patient may be measured prior to the treatment.

The method may further comprise providing the patient with psychotherapy. The psychotherapy treatment may be provided before and/or after the administration of the psychedelic compound.

The psychotherapy treatment may be provided before the administration of the psychedelic compound, after the administration of the psychedelic compound, during the administration of the psychedelic compound, or at any combination of two or more of before, during and after the administration of the psychedelic compound. Typically the psychotherapy treatment may be provided before, during and after the administration of the psychedelic compound. As used herein, providing psychotherapy treatment during the administration of the psychedelic compound means administering the psychedelic compound during a session of psychotherapy treatment.

The psychotherapy treatment may be provided in the three months before and/or the three months after administration of the psychedelic compound. Typically the psychotherapy treatment is provided in the two months before and/or the two months after administration of the psychedelic compound. The psychotherapy treatment may be provided in the eight weeks before and/or the eight weeks after administration of the psychedelic compound. Where the psychotherapy treatment is provided before and after the administration of the psychedelic compound, the psychotherapy treatment before the administration and the psychotherapy treatment after the administration may be provided in different timeframes. For example, the psychotherapy treatment may be provided in the three months before the administration and in the eight weeks after the administration. Alternatively, the psychotherapy treatment before the administration and the psychotherapy treatment after the administration may be provided in the same timeframe.

The patient may receive further psychotherapy in addition to the psychotherapy treatment that may be provided as part of the method of the invention. For example, the patient may receive further psychotherapy at least once a year following the administration of the psychedelic compound or the end of the psychotherapy treatment. The patient may receive the further psychotherapy at least twice a year, or at least four times a year.

The psychotherapy treatment may comprise at least 6 hours of psychotherapy treatment. Typically, the psychotherapy treatment comprises at least 8 hours, at least 10 hours, at least 12 hours or at least 20 hours of psychotherapy treatment. Where the psychotherapy treatment is provided before and after the administration of the psychedelic compound, the psychotherapy treatment before the administration may comprise the same number of hours as the psychotherapy treatment after the administration. For example, the psychotherapy treatment may comprise 6 hours of psychotherapy treatment before the administration and 6 hours of psychotherapy treatment after the administration. Alternatively, the psychotherapy treatment before the administration may comprise a different number of hours to the psychotherapy treatment after the administration. For example, the psychotherapy treatment may comprise 6 hours of psychotherapy treatment before the administration and 12 hours of psychotherapy treatment after the administration. Where the psychotherapy treatment is provided during the administration of the psychedelic compound and before and/or after the administration, the psychotherapy treatment during the administration may comprise the same number of hours as the psychotherapy treatment before and/or after the administration, or the psychotherapy treatment during the administration may comprise a different number of hours as the psychotherapy treatment before and/or after the administration. For example, the psychotherapy treatment may comprise 6 hours of psychotherapy treatment before the administration, 8 hours of psychotherapy treatment during the administration, and 6 hours of psychotherapy treatment after the administration.

The psychotherapy treatment may be provided in sessions at least 30 minutes in duration or at least one hour in duration. Typically, the psychotherapy treatment is provided in sessions at least two hours in duration. The psychotherapy treatment may comprise any number of sessions. Typically the psychotherapy treatment comprises at least 6 sessions. Where the psychotherapy treatment is provided before and after the administration of the psychedelic compound, the psychotherapy treatment before the administration may comprise the same number of sessions as the psychotherapy treatment after the administration. For example, the psychotherapy treatment may comprise 3 sessions before and 3 sessions after the administration. Alternatively, the psychotherapy treatment before the administration may comprise a different number of sessions to the psychotherapy treatment after the administration. For example, the psychotherapy treatment may comprise 3 sessions before and 6 sessions after the administration. Any psychotherapy treatment that is provided during the administration of the psychedelic compound is typically provided in a single session around 8 hours in duration.

The psychotherapy treatment may be any suitable psychotherapy technique. The psychotherapy treatment may comprise any psychotherapy delivered by two therapists. Typically the psychotherapy treatment is selected from dyadic psychotherapy, supportive psychotherapy, attachment-based psychotherapy, behavioural therapy, body psychotherapy, somatic psychotherapy, brief therapy, cognitive analytical therapy, cognitive behaviour therapy, existential psychotherapy, gestalt therapy, humanistic integrative psychotherapy, hypno-psychotherapy, Jungian analysis, neuro-linguistic psychotherapy, object relations therapy, person-centered psychotherapy, psychodynamic psychotherapy or psychoanalysis, solution-focused brief therapy, transactional analysis, family systems therapy, internal family systems therapy, transpersonal psychotherapy, emotion-focused therapy, compassion-focused therapy, mindfulness-based cognitive therapy. In one embodiment, the psychotherapy treatment is dyadic psychotherapy.

Dyadic psychotherapy is a known technique in psychedelic psychotherapy. As used herein, dyadic psychotherapy is psychotherapy administered by a dyad of two therapists. The dyadic psychotherapy treatment may include elements from supportive psychotherapy; cognitive-behavioral therapy; existentially oriented therapy (i.e. psychotherapies developed specifically by psycho-oncologists to address existentially oriented issues that arise in patients with cancer and especially advanced-staged diagnoses); and psychodynamic/psychoanalytic therapy. In one embodiment, the psychotherapy may be administered by a single therapist.

An advantageous effect of the present invention is that the psychedelic compound is useful in achieving rapid reductions in suicidal ideation and/or desire for hastened death. Therefore, in the method of the invention the suicidal ideation and/or desire for hastened death may be reduced within two weeks after administration of the psychedelic compound. In one embodiment, the desire for hastened death may be reduced within two weeks after administration of the psychedelic compound. Typically, the suicidal ideation and/or desire for hastened death is reduced within one week, within forty-eight hours, within one day (twenty-four hours), within twelve hours or within eight hours after administration of the psychedelic compound. In one embodiment, the suicidal ideation and/or desire for hastened death is reduced within twelve hours after administration of the psychedelic compound.

A further advantageous effect is that administration of a single dose of the psychedelic compound is useful in achieving sustained, long-term reductions in suicidal ideation and/or desire for hastened death. Thus, the psychedelic compound may be administered in a single dose. Where the psychedelic compound is administered in a single dose, no further dose may be administered for at least two weeks, or at least six months, or at least one year, or at least two years, or at least three years, or at least four and a half years, after administration of the single dose. Typically, where the psychedelic compound is administered in a single dose, no further dose is administered for at least six months or at least one year after administration of the single dose. In one embodiment, where the psychedelic compound is administered in a single dose, no further dose is administered for at least six months after administration of the single dose.

In one embodiment, a single dosage form (such as a tablet) comprising the psychedelic compound is administered to the patient in order to provide the single dose. However, as this skilled person will appreciate, this does not necessarily mean that only a single dosage form comprising the psychedelic compound may be administered. The single dose of the psychedelic compound may be administered as two or more separate dosage forms, which two or more separate dosage forms are administered simultaneously or shortly after one another (for instance less than 2 hours apart). The single dose may also be administered over a period of time, for example if being administered as an infusion. The single dose of the psychedelic compound is typically administered in a timeframe of no greater than about one hour.

In the method of the invention the suicidal ideation and/or desire for hastened death may be reduced or prevented for a period of at least six months, at least one year, at least three years, or at least four and a half years after administration of the psychedelic compound. In one embodiment, no further dose of the psychedelic compound is administered for at least six months after administration of the single dose. Typically, the suicidal ideation and/or desire for hastened death is reduced or prevented for at least six months or at least one year after administration of the single dose. In one embodiment, the suicidal ideation and/or desire for hastened death is reduced or prevented for at least six months after administration of the psychedelic compound, wherein no further dose of the psychedelic compound is administered for at least six months after administration of the single dose of the psychedelic compound. For example, the suicidal ideation may be reduced or prevented for a period of at least six months after administration of single dose. In another embodiment, the suicidal ideation and/or desire for hastened death is reduced or prevented for at least one year after administration of the single dose. For example, the desire for hastened death may be reduced or prevented for a period of at least one year after administration of the single dose.

The length of time for which no further dose of the psychedelic compound is administered after administration of the single dose may be the same as the length of time for which the suicidal ideation and/or desire for hastened death is reduced or prevented. Alternatively, the length of time for which no further dose of the psychedelic compound is administered after administration of the single dose of the psychedelic compound may be different to the length of time for which the suicidal ideation and/or desire for hastened death is reduced or prevented.

The treatment also advantageously has an effect which is both acute and sustained. Thus, the SI and/or DHD may be reduced within 24 hours and may remain reduced for at least 6 months.

The life-threatening disease may be any chronic disease which has the potential to reduce the normal life expectancy of a patient suffering from the disease. The life-threatening disease may be selected from cancer, heart disease, chronic obstructive pulmonary disease (COPD), diabetes mellitus, Alzheimer's, dementia, motor neurone disease, amyotrophic lateral sclerosis (ALS), Parkinson's disease, epilepsy, multiple sclerosis, and myalgic encephalopathy (ME). In one embodiment, the life-threatening disease is cancer.

As used herein, the term “patient suffering from a life-threatening disease” describes a human patient who has been diagnosed with having a life-threatening disease at the time of the administration of the psychedelic compound (e.g. psilocybin). In one embodiment, the patient suffering from a life-threatening disease is a cancer patient. The patient may have an active diagnosis of the life-threatening disease for as long as the suicidal ideation and/or desire for hastened death in said patient is reduced or prevented. Alternatively, where the life-threatening disease is one which may be cured, the patient may be cured of the disease during said reduction or prevention of suicidal ideation and/or reduction of prevention of desire for hastened death. For example, where the patient is a cancer patient, the cancer patient may enter partial or complete remission from cancer during said reduction or prevention of suicidal ideation and/or reduction or prevention of desire for hastened death.

Where the life-threatening disease is cancer, the cancer patient may be suffering from stage I, stage II, stage III or stage IV cancer. All stages, but in particular advanced stages, of cancer are associated with elevated rates of suicidal ideation and desire for hastened death. The cancer patient may be suffering from late-stage cancer. Typically, the cancer patient is suffering from stage III or stage IV cancer. Stage I cancer typically refers to cancer where the tumours are small and the cancer is contained with the organ it started in. Stage II cancer typically refers to cancer where the tumours are larger than in stage I, but the cancer has not yet started to spread to surrounding tissues. Stage III cancer typically refers to cancer with large tumours, where the cancer may have spread into the surrounding tissues and nearby lymph nodes. Stage IV cancer typically refers to metastatic cancer, where the cancer has spread from where it started to another organ.

The cancer may be selected from breast cancer, reproductive cancer, lymphoma, leukemia, acute lymphoblastic leukemia (ALL), acute myeloid leukemia (AML), adrenocortical carcinoma, anal cancer, appendix cancer, atypical teratoid/rhabdoid tumor, bile duct cancer, bladder cancer, bone cancer, brain cancer, Burkitt lymphoma, cervical cancer, chronic lymphocytic leukemia (CLL), chronic myelogenous leukemia (CML), chronic myeloproliferative neoplasms, colorectal cancer, cutaneous T-cell lymphoma, esophageal cancer, eye cancer, intraocular melanoma, fallopian tube cancer, gallbladder cancer, gastric (stomach) cancer, gastrointestinal carcinoid tumor, germ cell tumor, hairy cell leukemia, head and neck cancer, heart cancer, Hodgkin lymphoma, intraocular melanoma, islet cell tumor, kidney cancer, Langerhans cell histiocytosis, liver cancer, lung cancer (non-small cell, small cell, pleuropulmonary blastoma, and tracheobronchial tumor), melanoma, mesothelioma, metastatic cancer, mouth cancer, multiple endocrine neoplasia syndrome, multiple myeloma/plasma cell neoplasms, myelodysplastic syndrome, nasopharyngeal cancer, neuroblastoma, non-Hodgkin lymphoma, oral cancer, oropharyngeal cancer, osteosarcoma, ovarian cancer, pancreatic cancer, papillomatosis, paraganglioma, parathyroid cancer, penile cancer, pheochromocytoma, pituitary tumor, primary central nervous system (CNS) lymphoma, primary peritoneal cancer, prostate cancer, rectal cancer, retinoblastoma, salivary gland cancer, sarcoma, skin cancer, small intestine cancer, soft tissue sarcoma, testicular cancer, throat cancer, thymic carcinoma, thyroid cancer, urethral cancer, uterine cancer, and vaginal cancer.

The method of the invention is particularly useful where a cancer patient with said cancer is more likely to have suicidal ideation and/or desire for hastened death (for example because the cancer causes high levels of pain or causes a significant reduction in quality of life). Accordingly, the cancer may be selected from breast cancer, reproductive cancer, lymphoma, leukemia, bladder cancer, head and neck cancer, lung cancer and testicular cancer. In one embodiment, the cancer is selected from breast cancer, leukemia, lymphoma, and reproductive cancer.

In one embodiment, the patient identifies as male. In one embodiment, the patient identifies as female. In one embodiment, the patient identifies as non-binary. The patient may be from 21 to 80 years old. In one embodiment, the patient is from 40 to 60 years old. In one embodiment, the patient has not received a psychedelic compound at any point in his or her life prior to the administration of the psychedelic compound (i.e. the patient has not taken or received a psychedelic compound in his or her lifetime prior to the treatment of the invention). For instance, the patient may have never before been administered psilocybin (including self-administration). In another embodiment, the patient has received a psychedelic compound at some point in his or her life prior to the administration of the psychedelic compound in the method of the invention. For instance, the patient may have been administered psilocybin at a point in the past (e.g. prior to the diagnosis of the life-threatening disease). For instance, the patient may have been administered psilocybin more than one year, or more than five years, before the treatment of the invention.

The method of the invention may further comprise assessing the patient to determine the extent to which suicidal ideation and/or desire for hastened death has been reduced. The patient may be assessed within twelve hours of administration of the psychedelic compound. The patient may be assessed at least two weeks after the administration of the psychedelic compound. In one embodiment, the patient is assessed at least six months, at least two years, at least three years or at least four and a half years after the administration of the psychedelic compound.

The patient may have an anxiety-related diagnosis. Accordingly, the method of the invention may be used to reduce or prevent suicidal ideation and/or reduce or prevent desire for hastened death in patients who are also in need of treatment to alleviate anxiety and/or depression. Alternatively, the patient may not have an anxiety-related diagnosis. Accordingly, the method of the invention may be used to reduce or prevent suicidal ideation and/or reduce or prevent desire for hastened death in patients who are not in need of treatment to alleviate anxiety and/or depression.

The anxiety-related diagnosis may include an anxiety-related diagnosis as measured using the Diagnostic and Statistical Manual of Mental Disorders (DSM-IV). The anxiety-related diagnosis may be selected from an adjustment disorder, panic disorder, post-traumatic stress disorder, obsessive-compulsive disorder, social phobia, acute stress disorder, and generalized anxiety disorder. Typically, the anxiety-related diagnosis is an adjustment disorder or generalized anxiety disorder.

The psychedelic compound is administered in an amount effective to reduce or prevent suicidal ideation and/or reduce or prevent desire for hastened death. An effective amount of the psychedelic compound may be from about 0.001 mg/kg to about 10 mg/kg, for instance from about 0.01 mg/kg to about 1 mg/kg, where mg/kg is mg per kg of the patient's body weight at the time of the administration of the dose. Typically, an effective amount of the psychedelic compound may be a dose of from about 0.1 mg/kg to about 0.5 mg/kg, or from about 0.2 mg/kg to about 0.4 mg/kg. In one embodiment, the effective amount of the psychedelic compound is about 0.3 mg/kg.

The effective dose of the psychedelic compound may be from about 0.1 to about 100 mg. In one embodiment, the psychedelic compound is administered in a dose of from about 1 mg to 60 mg or from about 10 mg to about 40 mg. Typically, the dose may be from about 10 mg to about 35 mg, or from about 15 mg to about 30 mg, or from about 20 mg to about 30 mg. In one embodiment, the dose is about 25 mg. In one embodiment, the dose is from about 1 mg to about 10 mg.

As discussed above, the dose may be a single dose with no further dose administered for a period of time after the single dose. Alternatively, the psychedelic compound may be administered more than once. In one embodiment, the psychedelic compound is administered one, two, three or four times. The psychedelic compound may be administered one, two or three times a year. Each dose of the psychedelic compound may be accompanied by a psychotherapy treatment as described herein.

In one embodiment, the psychedelic compound is psilocybin. The method of the invention may accordingly comprise administering a dose of psilocybin which is from about 0.01 mg/kg to about 1 mg/kg. The dose of psilocybin may be from about 0.1 mg/kg to about 0.5 mg/kg, or from about 0.2 mg/kg to about 0.4 mg/kg. In one embodiment, the dose of psilocybin is about 0.3 mg/kg.

Alternatively, the method of the invention may comprise administering a dose of psilocybin which is from about 10 mg to about 40 mg. The dose of psilocybin may be from about 10 mg to about 35 mg, or from about 15 mg to about 30 mg, or from about 20 mg to about 30 mg. In one embodiment, the single dose of psilocybin is about 25 mg.

In one embodiment, the patient is not administered a separate treatment for the reduction or prevention of suicidal ideation and/or reduction or prevention of desire for hastened death for the period during which suicidal ideation and/or desire for hastened death is reduced or prevented.

Kits

The kit of the invention contains the psychedelic compound; and instructions for use of said psychedelic compound in the method of reducing or preventing suicidal ideation and/or desire for hastened death in a patient suffering from a life-threatening disease. Said instructions may include, for example, instructions for psychotherapy treatment to be provided before, during, and/or after the administration of the psychedelic compound. The psychedelic compound is typically in the form of a pharmaceutical formulation, for instance as described below.

Formulation of the Psychedelic Compound

The psychedelic compound may be administered to the patient suffering from a life-threatening disease by any acceptable route of administration including, but not limited to, inhaled, oral, nasal, topical (including transdermal) and parenteral modes of administration. The psychedelic compound may be administered as, for example: a tablet, capsule, powder, solution or suspension for oral administration; a solution or suspension for injection; or a solution, suspension or powder for inhalation. The psychedelic compound may be administered in a food stuff, for instance a food stuff comprising a natural source of the psychedelic compound such as a fungus of the genus Psilocybe.

Depending on the mode of administration used, the psychedelic compound (e.g. psilocybin) may be formulated with an appropriate conventional carrier, excipient or diluent. Pharmaceutically acceptable excipients, carriers and diluents are well known to the skilled person.

The diluent may be any pharmaceutically acceptable diluent. The diluent is typically suitable for parenteral administration or for oral administration. Examples of suitable liquid diluents include water, ethanol and glycerol. The diluent may alternatively be selected from solid diluents such as lactose, dextrose, saccharose, cellulose, corn starch and potato starch. The diluent may contain buffer components to control the pH. The buffers may be derived from phosphate, citrate or acetate. The diluent may also contain sodium chloride.

The excipient may be selected from: lubricants, e.g. silica, talc, stearic acid, magnesium or calcium stearate, and/or polyethylene glycols; binding agents, e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose, glycerine, mannitol or sorbitol.

Suspensions or emulsions may contain as carrier, for example a natural gum, agar, sodium alginate, pectin, methylcellulose, carboxymethylcellulose, or polyvinyl alcohol. The suspension or solutions for intramuscular injections may contain, together with the psychedelic compound, a pharmaceutically acceptable carrier, e.g. sterile water, olive oil, ethyl oleate, glycols, e.g. propylene glycol, and if desired, a suitable amount of lidocaine hydrochloride. Solutions for injection or infusion, or for inhalation, may contain as carrier, for example, sterile water or they may be in the form of sterile, aqueous, isotonic saline solutions.

The invention is described in more detail by the following Example. Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

The texts of references cited in this disclosure are herein incorporated by reference in their entireties.

### Example

The present example assesses the effects of a single dose of psilocybin (0.3 mg/kg) in conjunction with psychotherapy in the reduction of suicidal ideation and desire for hastened death of patients with a life-threatening disease, specifically cancer patients.

Methods

29 participants were randomly assigned to one of two groups: psilocybin (0.3 mg/kg) on the first medication session followed by niacin (250 mg) on the second session (i.e. psilocybin-first group), or niacin (250 mg) on the first medication session followed by psilocybin (0.3 mg/kg) on the second session (i.e. niacin-first group). Psilocybin and niacin were administered in identically appearing gelatin capsules.

Participants received three 2-hour preparatory psychotherapy sessions in order to review the purpose and intention of participation in the study, treatment goals, and structure of the treatment sessions. Medications were administered in 8-hour treatment sessions which included a discussion of the participant's subjective experience with the treatment team to consolidate the memory of the experience and to begin the process of post-integrative psychotherapy. Participants also received three 2-hour post-medication integration sessions (repeated after the second dosing session) to further consolidate the memory of the experience and to continue the process of psychological integration. All psychotherapy treatment sessions were delivered by a dyadic therapy team trained in a blend of psychotherapies with an evidence base to support their use in cancer patients (drawing upon principles of palliative care therapy, existential psychotherapy, cognitive-behavioural therapy, psychoanalysis therapy, and transpersonal psychology) and were taught about the safety and clinical pharmacology of psilocybin. Each therapy dyad underwent six confidential one-to-one sessions to build their alliance and exchange ideas about psychedelic therapy. The trial employed a crossover design at seven weeks following the first drug administration, with an outcome assessment at 6.5 months (26 weeks) following the second drug administration (i.e. after the crossover).

Two long-term follow ups (LTFUs) were carried out to assess the continued effect of psilocybin. Of the original 29 participants, all 16 participants who were not deceased at the time of LTFU were contacted (the remaining 13 participants were deceased). Of the 16 participants who were contacted, 15 agreed to participate in the LTFU and completed measures through a secure online portal. One participant died (from cancer-related complications) after completing the first LTFU and prior to the second LTFU, leaving 14 participants at the second LTFU. The first and second LTFUs occurred on average 3.2 years (range 2.3-4.5 years) and 4.5 years (range 3.5-5.5 years) following the participants' psilocybin dosing date, respectively.

Participants

Demographic information is presented in Table 1. The mean age of participants was 60.3 years old (standard deviation (SD)=7.1 years), and they were predominately female (63.6%). The majority was non-Hispanic White (90.9%), followed by Multiracial (9.1%). Approximately 46% reported identifying as Atheist/Agnostic versus some other organized religious affiliation. Gynecological cancers (54.6%) comprised the majority of disease sites. Nearly three-quarters (72.8%) were diagnosed with advanced [111-IV] versus early [I-Il] stage (27.2%) cancers.

Approximately one third (36.4%) of all participants reported one or more occasions of prior psychedelic use. The majority of participants met Diagnostic and Statistical Manual of Mental Disorders (DSM-IV; American Psychiatric Association, 2000) criteria for cancer-related adjustment disorder with anxious and depressed features (45.5%) or adjustment disorder with anxious (only) features (45.5%), followed by generalized anxiety disorder (9.1%).

TABLE 1
Demographic and clinical characteristics of study participants included in present study

| | | Psilocybin | | Niacin | | | |
|---|---|---|---|---|---|---|---|
| | | first | | first | | Total | |
| Characteristic | Categories | n = 6 | | n = 5 | | N = 11 | |
| Sex | Female | 3 | 50% | 4 | 80% | 7 | 63.6% |
| | Male | 3 | 50% | 1 | 20% | 4 | 36.4% |
| Age at follow- | Range 48-71 | 57.5 (7.5) | | 63.6(5.3) | | 60.3 (7.1) | |
| up, mean (SD) | | | | | | | |
| Race | White/Caucasian | 5 | 100% | 4 | 80% | 10 | 90.9% |
| | Multiracial | 0 | 0% | 1 | 20% | 1 | 9.1% |
| Religious/ | Atheist/Agnostic | 1 | 16.7% | 4 | 80% | 5 | 45.5% |
| spiritual beliefs | Jewish | 1 | 16.7% | 0 | 0% | 1 | 9.1% |
| | Catholic | 2 | 33.3% | 0 | 0% | 2 | 18.2% |
| | Unitarian | 1 | 16.7% | 0 | 0% | 1 | 9.1% |
| | Other faith/tradition | 1 | 16.7% | 1 | 20% | 2 | 18.2% |
| Site of cancer | Breast | 0 | 0% | 1 | 9.1% | 1 | 9.1% |
| | Reproductive | 3 | 50% | 2 | 18.2% | 5 | 45.5% |
| | Lymphoma/Leukemia | 0 | 0% | 1 | 9.1% | 1 | 9.1% |
| | Colon | 0 | 0% | 1 | 9.1% | 1 | 9.1% |
| | Other types | 3 | 50% | 0 | 0% | 3 | 27.3% |
| Stage of cancer | Stage IV | 2 | 40% | 1 | 16.7% | 3 | 27.3% |
| | Stage III | 2 | 40% | 5 | 45.5% | 5 | 45.5% |
| | Stage II | 1 | 20% | 0 | 0% | 1 | 9.1% |
| | Stage I | 0 | 0% | 2 | 33.3% | 2 | 18.2% |
| SCID (DSM-IV) | Adjustment disorder w/anxiety and | 2 | 33.3% | 3 | 60% | 5 | 45.5% |
| diagnosis | depressed mood, chronic | | | | | | |
| | Adjustment disorder w/anxiety, | 3 | 50% | 2 | 40% | 5 | 45.5% |
| | chronic | | | | | | |
| | Generalized Anxiety Disorder | 1 | 16.7% | 0 | 0% | 1 | 9.1% |
| Previous | No | 4 | 66.7% | 3 | 60% | 7 | 63.6% |
| Psychedelic | | | | | | | |
| Use | Yes | 2 | 33.3% | 2 | 40% | 4 | 36.4% |
| Education | Grade 7-12 w/o graduating High | 1 | 16.7% | 0 | 0% | 1 | 9.1% |
| | School | | | | | | |
| | Part-college | 1 | 16.7% | 1 | 20% | 2 | 18.2% |
| | Graduated 4-year college | 2 | 33.3% | 2 | 40% | 4 | 36.4% |
| | Completed grad/professional school | 2 | 33.3% | 2 | 40% | 4 | 36.4% |

Psychiatric Interventions Received During Follow-Up Period

Only 8% of participants reported receiving any psychotherapy or pharmacotherapy specifically targeting cancer-related psychiatric distress over the ensuing period from the end of the parent study to the final LTFU assessment.

Outcome Measures

Primary outcomes (suicidal ideation and desire for hastened death) were measured according to the following methods:
Beck Depression Inventory-II (BDI-II) (Beck et al., 1988)— this is a widely used self-report measure to assess depression. The BDI-II comprises 21 questions related to depressive symptoms experienced over the previous two weeks, and is rated on a 3-point scale (total score ranges from 0-63). Scores greater than 12 correlate with clinical depression. This measure demonstrates good reliability (internal consistency 0.90) and factorial validity (Storch et al. 2004). Item #9 on the BDI queries suicidal ideation with the following options: 0=I don't have any thoughts of killing myself; 1=I have thoughts of killing myself, but I would not carry them out; 2=I would like to kill myself; 3=I would kill myself if I had the chance. The BDI-II was assessed at baseline, 1-day prior to dose-1, 8-hours after dose-1, 1-day after dose-1, 2-weeks after dose-1, 6-weeks after dose-1, 7-weeks after dose-1 (corresponding to 1-day prior to dose-2), 1-day after dose-2, 6-weeks after dose-2, 26-weeks after dose-2, and at the 3.2 year and 4.5 year LTFU assessments.
The Brief Symptom Inventory (BSI) (Derogatis 1993) is a 53-item self-report questionnaire that assesses a range of psychiatric symptoms, generating 9 domains (depression, anxiety, phobic anxiety, somatization, obsession-compulsion, interpersonal sensitivity, hostility, paranoid ideation, psychoticism) and a global severity index. It is measured on a Likert scale: 0=Not at all; 1=Little, 2=Moderately; 3=Quite a bit; 4=Extremely. There is one item that directly relates to suicidal ideation: Item #9 (“Thoughts of ending your life”). The BSI was were assessed at baseline, 1-day prior to dose-1, 8-hours after dose-1, 1-day after dose-1, 2-weeks after dose-1, 6-weeks after dose-1, 7-weeks after dose-1 (corresponding to 1-day prior to dose-2), 1-day after dose-2, 6-weeks after dose-2, and 26-weeks after dose-2, but not at the 3.2 year and 4.5 year LTFU assessments.
Composite SI score: A composite score of SI [BDI-II item #9+BSI Item #9] was created to decrease the number of statistical tests and the potential for Type II error. Because the BDI-II and BSI utilize disparately weighted Likert scales, the composite score was calculated by computing Z-scores for each item and summing them. To enhance interpretation, the composite Z-scores were transformed into standardized T-scores with a range of 0 to 100 (Song et al., 2013). Higher scores indicate higher SI. Since only the BDI-II was administered at the LTFU time points, but not the BSI, a composite SI score was not generated at the two LTFU assessments with the final composite SI score occurring at the final 6.5 month follow-up assessment from the original/parent study.
The Demoralization Scale (DS) (Kissane et al., 2004) is a 24-item questionnaire that was developed to measure existential distress in advanced cancer. The DS generates five factors: loss of meaning, despair, disheartened feelings, helpless feelings, and a sense of failure. Likert scale items range from zero to four, and total scores range from zero to 96. Scores above 30 are considered indicative of clinical levels of demoralization. This measure has shown good reliability (Cronbach's a ranging from 0.71 to 0.89) and concurrent validity, with regard to related scales (Kissane et al. 2004). The DS was assessed at baseline, 2-weeks post dose-1, 26-weeks post dose-2, and at the 3.2 year and 4.5 year LTFU assessments.
Composite DHD score: Since the loss of meaning factor from the DS is most closely correlated with the schedule of attitudes towards hastened death SAHD scale (Kissane et al. 2004), in this analysis the following 5 items from the loss of meaning factor were added together to generate a composite score that functioned as a proxy for DHD: “Life is no longer worth living”, “I would rather not be alive”, “My life seems to be pointless”, “My role in life has been lost”, and “There is no purpose to the activities in my life”.

Data Analysis

Data were drawn from the original parent investigation and the LTFU study for secondary analyses. Of the original 29 participants, a subset of 11 participants [psilocybin 1^{st}/Niacin 2^{nd} (N=6); Niacin 1st/Psilocybin 2^{nd} (N=5)] who met criteria for having a baseline level of suicidality (defined as a composite SI score >0 on BDI item 9+BSI item 9) were included in the present analysis. While this approach reduces power by limiting the sample size, it addresses concerns for a floor effect and more optimally addresses the main study objective of this secondary analysis as to whether psilocybin therapy reduces suicidality.

Repeated measures regressions, from the mixed effect repeated measurement (MMRM) model, were performed in SPSS v25 using an AR(1) covariance structure and fixed effects of group and time. Planned comparison between group (psilocybin 1st vs niacin 1st) t tests from the MMRM analyses were conducted to assess the composite SI score at the following time points: baseline (2-4 weeks prior to dose-1), 8-hours after dose-1, 2-weeks after dose-1, and 7-weeks after dose-1 (1-day prior to dose-2/cross-over). In addition, planned comparison between group (psilocybin 1^{st} vs niacin 1^{st}) t tests from the MMRM analyses were conducted to assess the composite DHD score at the following time points: baseline and 2-weeks after dose-1. Between-group effects were calculated using Cohen's d.

After the cross-over, both the psilocybin 1^{st} and niacin 1^{st} dose sequence groups were collapsed and combined into one group. This approach was chosen because the crossover design prevented valid between-group comparisons subsequent to the crossover, and to increase power given the modest sample size. The long-term effects of psilocybin on variables of interest were assessed with the MMRM model. Planned within-subject t-tests (Tukey's post-hoc) were conducted comparing scores at baseline to the following time points after the 2^{nd} medication session: 6.5-months, 3.2 years, and 4.5 years. Within-group effect sizes were calculated using Cohen's d. Note: the BDI (and not the BSI) was re-administered at 3.2 years and 4.5 years so the composite SI score was not available for analysis at the LTFU time points. The DHD composite score was available at both LTFU assessments.

Change scores were calculated for composite SI (with transformed T-scores) by subtracting baseline scores from scores at 2-week post dose-1. Pearson correlation coefficients between changes in composite SI were also calculated at 2-weeks post dose-1.

Results

(i) Primary Outcomes

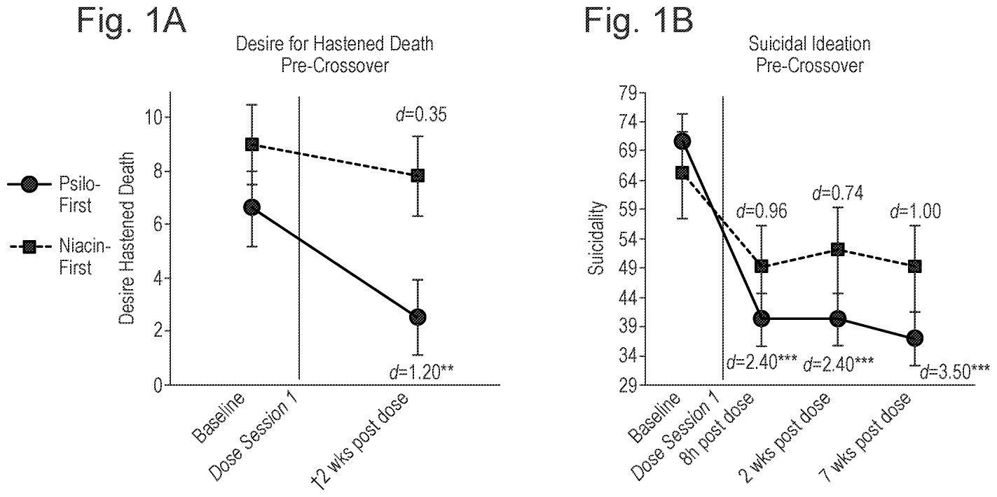

On the DHD, MMRM analyses indicated that there was no significant omnibus interaction of group and time (F_{(4, 27)}=0.67 p=0.429); however, there was a main effect of group (F_{(1, 11)}=6.49, p=0.027) and time (see details below). Pairwise comparisons specified a priori indicated that mean scores on the DHD differed significantly between the psilocybin-1^{st} and niacin-1^{st} groups at 2-weeks following the first drug administration session (p=0.021). The magnitude of this between-group difference was large (Cohen's d=1.32) (see FIG. 1A). On the composite SI measure, a significant interaction of group and time was not detected (p=0.065), nor any main effect of group (p=0.614), indicating no significant differences between the two groups prior to the crossover (see FIG. 1B).

Within the psilocybin-1^{st} group, MMRM analyses indicated significant main effects of time on DHD (F_{(4,18)}=5.60, p=0.004) and SI (F_{(4, 23)}=9.55, p<0.001). Prior to the crossover, pairwise comparisons specified a priori indicated that mean scores differed significantly at all post dose-1 administration session time points relative to baseline on DHD at 2-weeks post dose-1 (p=0.005) (see FIG. 1A), and SI at 8-hours (p<0.001, d=2.40), 2-weeks (p<0.001, d=2.40), and 7-weeks (p<0.001, d=3.50) post-dose 1 (see FIG. 1B). Within-subject effect sizes across these measures and time points for statistically significant reductions were large (Cohen's d range=1.20-3.50). Prior to the crossover in the niacin-1^{st} group, there was no significant main effect of time on the composite DHD score (F(4,10)=9.64, p=0.079) or the composite suicidality/SI score (F(4,19)=2.64, p=0.065) (see FIGS. 1A and 1B), indicating no significant within-group reductions.

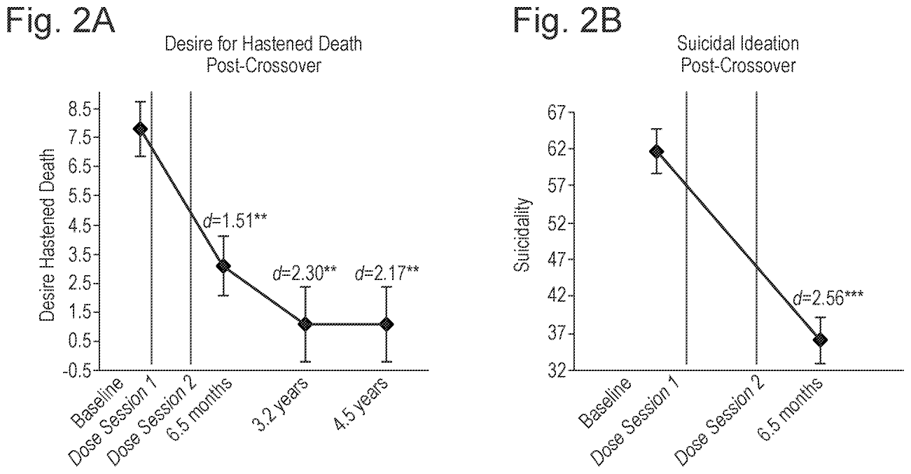

After the crossover, with both dose-sequence groups collapsed and combined into one group, statistically significant within-subjects reductions were detected, relative to baseline, on DHD (p<0.001) (see FIG. 2A) and SI (p<0.001) (see FIG. 2B) at the 6.5-month follow-up. Reductions in DHD remained significant at the 3.2 (p<0.001) and 4.5-year (p<0.001) follow-ups (see FIG. 2A). The composite SI measure was not administered at either of these long-term follow-up points. Within-subjects effect sizes across these measures and time points were large (Cohen's d range=1.51-2.56) (see FIGS. 2A and 2B).

### CONCLUSION

This secondary analysis included 11 participants (out of the total original sample of 29) who met criteria for having a baseline level of suicidality >0. This approach was chosen to counteract potential floor effects and better address the question of whether psilocybin therapy reduces suicidality in a trial where active suicidal ideation and behaviors were excluded and without a focus on recruiting individuals with some degree of suicidality.

The results show that a single moderate to high dose psilocybin, in conjunction with psychotherapy, produces acute (8 hours), post-acute (2 weeks) and sustained (i.e. 7-weeks to 6.5 months to 4.5 years) reductions in DHD and SI in patients with life-threatening cancer. The most compelling finding detected is a significant pairwise comparison indicating that mean scores on the DHD differed significantly between the psilocybin-1^{st} and niacin-1^{st} groups prior to the crossover at 2-weeks post dose-1. The data after the crossover, with both dose-sequence groups combined, revealed statistically significant, substantial [e.g., large within group effect sizes], and sustained improvements in SI [up to 6.5 months] and DHD [at 3.2 and 4.5 years] post dose-1, respectively.

### REFERENCES

Beck et al (1988), “Psychometric properties of the Beck Depression Inventory: Twenty-five years of evaluation”, Clin Psych Rev, 8: 77-100.
Carhart-Harris et al. (2018) “Psilocybin with psychological support for treatment-resistant depression: six-month follow-up”, Psychopharmacology (Berl), 235: 399-408.
Derogatis L. (1993), BSI. Administration, scoring and procedures manual for the brief symptom inventory (4th ed.), Minneapolis, MN: National Computer Systems.
Fergusson et al. (2005), “Association between suicide attempts and selective serotonin reuptake inhibitors: systematic review of randomised controlled trials”, British Medical Journal, 330.
Griffiths et al. (2016), “Psilocybin produces substantial and sustained decreases in depression and anxiety in patients with life-threatening cancer: A randomized double-blind trial”, J Psychopharmacol, 30: 1181-1197.
Hendricks P et al. (2015), “Psilocybin, psychological distress, and suicidality”, J Psychopharmacol, 29(9): 1041-1043.
Kissane et al. (2004), “The demoralization scale: A report of its development and preliminary validation”, J Palliat Care, 20: 269-276.
Rosenfeld B et al. (2000), “The schedule of attitudes toward hastened death: Measuring desire for death in terminally ill cancer patients”, Cancer, 88: 2868-2875.
Ross S et al. (2016), “Rapid and sustained symptom reduction following psilocybin treatment for anxiety and depression in patients with life-threatening cancer: A randomized controlled trial”, J Psychopharmacol, 30: 1165-1180.
Song M-K, et al. (2013) “Composite variables: when and how”, Nursing research 62(1): 45-49.
Stone M et al. (2009) “Risk of suicidality in clinical trials of antidepressants in adults: analysis of proprietary data submitted to US Food and Drug Administration”, British Medical Journal, 339.
Storch et al (2004), “Factor structure, concurrent validity, and internal consistency of the beck depression inventory—second edition in a sample of college students”, Depress Anxiety, 19: 187-189.

1. A method of preventing or reducing suicidal ideation and/or desire for hastened death in a patient suffering from a life-threatening disease,
the method comprising administering a psychedelic compound which is psilocin or a prodrug of psilocin, or a pharmaceutically acceptable salt thereof, to said patient.

2. The method of claim 1, wherein the method is a method of preventing or reducing suicidal ideation in a patient suffering from a life-threatening disease.

3. The method of claim 1, wherein the method is a method of preventing or reducing desire for hastened death in a patient suffering from a life-threatening disease.

4. The method of any one of claims 1 to 3, wherein the psychedelic compound is psilocybin or a pharmaceutically acceptable salt thereof.

5. The method of any one of the preceding claims, wherein the method further comprises providing the patient with psychotherapy treatment.

6. The method of claim 5, wherein the psychotherapy treatment is provided in the three months before and/or the three months after administration of the psychedelic compound.

7. The method of claim 5 or claim 6, wherein the psychotherapy treatment is selected from dyadic psychotherapy, supportive psychotherapy, attachment-based psychotherapy, behavioural therapy, body psychotherapy, somatic psychotherapy, brief therapy, cognitive analytical therapy, cognitive behaviour therapy, existential psychotherapy, gestalt therapy, humanistic integrative psychotherapy, hypno-psychotherapy, Jungian analysis, neuro-linguistic psychotherapy, object relations therapy, person-centered psychotherapy, psychodynamic psychotherapy or psychoanalysis, solution-focused brief therapy, transactional analysis, family systems therapy, internal family systems therapy, transpersonal psychotherapy, emotion-focused therapy, compassion-focused therapy, mindfulness-based cognitive therapy.

8. The method of claim 7, wherein the psychotherapy treatment is dyadic psychotherapy.

9. The method of any one of the preceding claims, wherein the patient suffering from a life-threatening disease is a cancer patient.

10. The method of claim 9, wherein the cancer patient is suffering from stage I or stage II cancer.

11. The method of claim 9, wherein the cancer patient is suffering from late-stage cancer.

12. The method of claim 9, wherein the cancer patient is suffering from stage III or stage IV cancer.

13. The method of any one of claims 9 to 12, wherein the cancer is selected from breast cancer, reproductive cancer, lymphoma, leukemia, acute lymphoblastic leukemia (ALL), acute myeloid leukemia (AML), adrenocortical carcinoma, anal cancer, appendix cancer, atypical teratoid/rhabdoid tumor, bile duct cancer, bladder cancer, bone cancer, brain cancer, Burkitt lymphoma, cervical cancer, chronic lymphocytic leukemia (CLL), chronic myelogenous leukemia (CML), chronic myeloproliferative neoplasms, colorectal cancer, cutaneous T-cell lymphoma, esophageal cancer, eye cancer, intraocular melanoma, fallopian tube cancer, gallbladder cancer, gastric (stomach) cancer, gastrointestinal carcinoid tumor, germ cell tumor, hairy cell leukemia, head and neck cancer, heart cancer, Hodgkin lymphoma, intraocular melanoma, islet cell tumor, kidney cancer, Langerhans cell histiocytosis, liver cancer, lung cancer (non-small cell, small cell, pleuropulmonary blastoma, and tracheobronchial tumor), melanoma, mesothelioma, metastatic cancer, mouth cancer, multiple endocrine neoplasia syndrome, multiple myeloma/plasma cell neoplasms, myelodysplastic syndrome, nasopharyngeal cancer, neuroblastoma, non-Hodgkin lymphoma, oral cancer, oropharyngeal cancer, osteosarcoma, ovarian cancer, pancreatic cancer, papillomatosis, paraganglioma, parathyroid cancer, penile cancer, pheochromocytoma, pituitary tumor, primary central nervous system (CNS) lymphoma, primary peritoneal cancer, prostate cancer, rectal cancer, retinoblastoma, salivary gland cancer, sarcoma, skin cancer, small intestine cancer, soft tissue sarcoma, testicular cancer, throat cancer, thymic carcinoma, thyroid cancer, urethral cancer, uterine cancer, and vaginal cancer.

14. The method of claim 13, wherein the cancer is selected from breast cancer, reproductive cancer, lymphoma, leukemia, bladder cancer, head and neck cancer, lung cancer and testicular cancer.

15. The method of claim 14, wherein the cancer is selected from breast cancer, leukemia, lymphoma, and reproductive cancer.

16. The method of any one of the preceding claims, wherein the suicidal ideation and/or desire for hastened death is reduced within twelve hours or within twenty-four hours after administration of the psychedelic compound.

17. The method of any one of the preceding claims, wherein the psychedelic compound is administered as a single dose, and wherein no further dose is administered for at least six months after administration of the single dose.

18. The method of claim 17, wherein the suicidal ideation and/or desire for hastened death is reduced or prevented for at least six months after administration of the single dose of the psychedelic compound.

19. The method of any one of the preceding claims, wherein the method comprises
assessing the level of suicidal ideation and/or desire for hastened death in the patient; and
administering a psychedelic compound which is psilocin or a prodrug of psilocin, or a pharmaceutically acceptable salt thereof, to said patient.

20. The method of any one of the preceding claims, wherein a composite suicidal ideation score of the patient is reduced after administration of the psychedelic compound.

21. The method of claim 20, wherein the composite suicidal ideation score of the patient is reduced by at least 30% after administration of the psychedelic compound.

22. The method of claim 1, wherein a composite desire for hastened death score of the patient after administration of the psychedelic compound is reduced compared to the composite desire for hastened death score of the patient before the administration.

23. The method of claim 22 wherein the composite desire for hastened death score of the patient is reduced by at least 40% after administration of the psychedelic compound.

24. The method of any one of the preceding claims, wherein the psychedelic compound is administered at a dose of from about 10 mg to about 40 mg.

25. The method of claim 24, wherein the psychedelic compound is administered at a dose of from about 20 mg to about 30 mg.

26. The method of claim 25, wherein the psychedelic compound is administered at a dose of about 25 mg.

27. A kit comprising:
a psychedelic compound which is psilocin or a prodrug of psilocin, or a pharmaceutically acceptable salt thereof; and
instructions for use of said psychedelic compound in a method of preventing or reducing suicidal ideation and/or preventing or reducing desire for hastened death in a patient suffering from a life-threatening disease, the method comprising administering the psychedelic compound to said patient.

28. A psychedelic compound which is psilocin or a prodrug of psilocin, or a pharmaceutically acceptable salt thereof, for use in preventing or reducing suicidal ideation and/or desire for hastened death in a patient suffering from a life-threatening disease.

29. Use of a psychedelic compound which is psilocin, or a prodrug of psilocin, or a pharmaceutically acceptable salt thereof, in the preparation of a medicament for use in preventing or reducing suicidal ideation and/or desire for hastened death in a patient suffering from a life-threatening disease.
